(19) United States
(12) Patent Application Publication
(10) Pub. No.: US 2023/0025193 A1
(43) Pub. Date: Jan. 26, 2023

(54) SYSTEM HAVING A TUBE SHAFT IMPELLER AND AN ASSOCIATED METHOD THEREOF

(71) Applicant: GLOBAL LIFE SCIENCES SOLUTIONS USA LLC, Marlborough, MA (US)

(72) Inventors: Kandakumar Murugesan, Hoodi Village, Bengaluru (IN); Nagaraj Raghavendra Rao, Hoodi Village, Bengaluru (IN); Saravanan Balakrishnan, Hoodi Village, Bengaluru (IN)

(21) Appl. No.: 17/779,986

(22) PCT Filed: Dec. 7, 2020
(86) PCT No.: PCT/EP2020/084922

§ 371 Date: May 25, 2022

(30) Foreign Application Priority Data
Dec. 23, 2019 (IN) 201941053347

(51) Int. Cl. B01F 27/2122; B01F 27/093; B01F 27/113; B01F 27/808; B01F 27/88; B01F 27/91; B01F 35/513; C12M 1/00; C12M 1/06; C12M 1/12

(52) U.S. Cl. CPC B01F 27/2122; B01F 27/093; B01F 27/113; B01F 27/808; B01F 27/88; B01F 27/91; B01F 35/513; C12M 23/14; C12M 27/02; C12M 23/26; C12M 23/06; B01F 2035/351

(57) ABSTRACT

A device (30) includes a base connector (32) having an opening (33) and an impeller connector (64) coupled to the base connector (32). The impeller connector (64) has a through-passage (66) aligned with the opening (33) of the base connector (32). Further, the device (30) includes a flexible tube (34) having a first end (36) and a second end (40), where the first end (36) of the flexible tube (34) is coupled to the impeller connector (64). Furthermore, the device (30) includes a seal component (38) and an impeller (42) coupled to the second end (40) of the flexible tube (34). Additionally, the device (34) includes an enclosure (46) disposed enclosing the impeller (42), the flexible tube (34), the impeller connector (64), and the base connector (32).

### TECHNICAL FIELD OF THE INVENTION

The present disclosure relates to impeller systems, and more particularly, to a system having a tube shaft impeller and an associated method of using such a system. Furthermore, more specifically, a bioreactor system having a tube shaft impeller is disclosed.

### BACKGROUND

A bioreactor is used to process biological materials (for example, to grow plant, animal cells, or the like) including, for example, mammalian, plant or insect cells and microbial cultures. Such devices may also be used for sterile mixing as well as non-sterile mixing applications. Some traditional bioreactors are designed as stationary pressurized vessels which can be mixed by several alternative means. Some other traditional bioreactors are designed as disposable bioreactors which utilize plastic sterile bags instead of a culture vessel made from stainless steel or glass.

Rocker bioreactor is a type of reactor having a platform on which a vessel/bag is placed, which provides movement around one or more axes by using an electrical motor. The rocker bioreactor generates a low shear environment for cells, as the cells are not directly exposed to fast moving tips of impeller blades. However, the rocking process is limited and cannot be utilized in a quick and efficient manner. Specifically, the rocking motion is limited to a low number of back and forth movements so as not to stress the system. Stirred tank bioreactors (STBRs) are reactors in which mixing has been accomplished in pressurized vessels/bags by internal mechanical agitation using impeller devices. The impeller must provide sufficiently rapid agitation to disperse all compounds and achieve an effectively homogeneous concentration inside the bioreactor. Single use STBRs typically use a flexible plastic bag as a reactor vessel enclosed by a stainless-steel support vessel. The agitation is typically provided by a magnetically driven rotating impeller.

Conventional bioreactors using impeller devices typically use magnetic stirrers for mixing within pressurized vessels. A magnetic stirrer-based bioreactor is not suitable for a microcarrier culture due to construction of the stirrer using bearings and shaft which churns shear-sensitive microcarriers. Further, such a stirrer generates higher friction and there are chances of contamination of culture medium due to impeller parts. Hence, such a stirrer is not completely aseptic.

### SUMMARY

In accordance with one embodiment, a device is disclosed. The device includes a base connector having an opening and an impeller connector coupled to the base connector. The impeller connector has a through-passage aligned with the opening of the base connector. Further, the device includes a flexible tube having a first end and a second end, wherein the first end of the flexible tube is coupled to the impeller connector. Furthermore, the device includes a seal component and an impeller coupled to the second end of the flexible tube. Additionally, the device includes an enclosure disposed enclosing the impeller, the flexible tube, the impeller connector, and the base connector.

In accordance with another embodiment, a system is disclosed. The system includes a base module having a base support and an impeller drive unit disposed within the base support. Further, the system includes a drive shaft having a straight portion and a bend portion, wherein the straight portion is directly coupled to the impeller drive unit. Furthermore, the system includes the device having a base connector having an opening and an impeller connector coupled to the base connector. The impeller connector has a through-passage aligned with the opening of the base connector. Further, the device includes a flexible tube having a first end and a second end, wherein the first end of the flexible tube is coupled to the impeller connector. Furthermore, the device includes a seal component and an impeller coupled to the second end of the flexible tube. Additionally, the device includes an enclosure disposed enclosing the impeller, the flexible tube, the impeller connector, and the base connector.

In accordance with yet another embodiment, a method is disclosed. The method includes driving an impeller by an impeller drive unit of a base module via a drive shaft. The drive shaft includes a straight portion and a bend portion, wherein the straight portion is directly coupled to the impeller drive unit. The base module further includes a base connector coupled to an impeller connector which is further coupled to a first end of a sealed flexible tube. The drive shaft extends through an opening of the base connector, a through-passage of the impeller connector, and the flexible tube. The impeller is coupled to the bend portion of the drive shaft via a second end of the sealed flexible tube. The method further includes stirring a medium filled inside an enclosure, by the impeller. A portion of the sealed flexible tube enclosing the bend portion of the drive shaft rotates along with the impeller and the drive shaft.

### BRIEF DESCRIPTION OF THE DRAWINGS

FIG. 1 shows a schematic perspective view of a base module and a vessel of a system, for example, a bioreactor according to one embodiment of the present disclosure;

FIG. 2 shows a schematic perspective view of a device of the system shown in FIG. 1 according to one embodiment of the present disclosure;

FIG. 3 shows a schematic perspective view of the device with a drive shaft according to one embodiment of the present disclosure;

FIG. 4 shows a partial schematic perspective view of the device according to one embodiment of the present disclosure;

FIG. 5 shows a partial schematic perspective view of a flexible tube and an impeller connector according to one embodiment of the present disclosure;

FIG. 6 shows a schematic perspective view of the system according to embodiments of FIGS. 1-5 of the present disclosure; and

FIG. 7 is a partial schematic perspective view of the system 10 according to the embodiments of FIGS. 1-6 of the present disclosure.

### DETAILED DESCRIPTION OF EMBODIMENTS

In accordance with the embodiments of the present disclosure, a device is disclosed. The device includes a base connector having an opening and an impeller connector coupled to the base connector. The impeller connector has a through-passage aligned with the opening of the base connector. The device further includes a flexible tube having a first end and a second end, wherein the first end of the flexible tube is coupled to the impeller connector. The device also includes a seal component and an impeller coupled to the second end of the flexible tube. Further, the device includes an enclosure disposed enclosing the impeller, the flexible tube, the impeller connector, and the base connector. In accordance with another embodiment of the present disclosure, a system having a base module and the exemplary device is disclosed. In accordance with yet another embodiment, a method for operating the system having the base module and the exemplary device is disclosed.

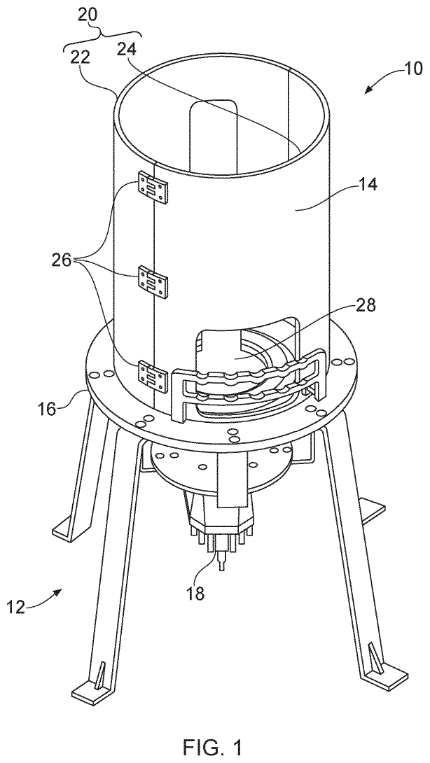

FIG. 1 shows a schematic perspective view of a base module 12 and a vessel 14 of an exemplary system 10 according to one embodiment of the present disclosure. In the illustrated embodiment, the system 10 is a bioreactor. The system 10 includes the base module 12 and the vessel 14 which are configured to support and substantially enclose an enclosure (not shown in FIG. 1). In one embodiment, the enclosure is a bag, for example, a bioreactor bag. In the illustrated embodiment, the vessel 14 is shown in a closed position. The size of the vessel 14 may vary depending on the application. In another embodiment, the enclosure is a container, for example, a metal container such as a stainless-steel container. In such an embodiment, the vessel 14 may not be required.

The base module 12 includes a base support 16 and an impeller drive unit 18 disposed within the base support 16. The vessel 14 includes a mating connection device (not shown) coupled to a corresponding mating connection device (not shown) of the base support 16. In one embodiment, a lower edge of the vessel 14 may be coupled to a groove (not shown) formed in the base support 16. Hence, the vessel 14 is stably supported by the base support 16. The vessel 14 includes a cylindrical side wall 20 having a first side wall 22 and a second side wall 24 coupled to each other via a plurality of hinges 26. The second side wall 24 can be opened to access interior of the vessel 14 and for loading and unloading the enclosure. The diameter of the cylindrical side wall 20 may vary depending on the application. In another embodiment, the vessel 14 may have a single integrated cylindrical side wall instead of a plurality of side walls.

Each of the first and second side walls 22, 24 may also include an opening (for example, opening 28) for providing access to the interior of the vessel 14. In one embodiment, the first and second side walls 22, 24 may be manufactured by plastic injection molding. In one specific embodiment, thermoplastic material can be used for molding the first and second side walls 22, 24 of the vessel 14. In another embodiment, the first and second side walls 22, 24 of the vessel 14 may be formed by stamping sheet metal or by 3D printing of either plastic or metal.

Furthermore, with reference to the plurality of hinges 26, opposite side edges of the first and second side walls 22, 24 is provided with a locking device (not shown) so that the first and second side walls 22, 24 can be detachably locked in a closed position. In one embodiment, the locking device may include co-operating magnets provided on side edges of both the first and the second side walls 22, 24. In another embodiment, the locking device may include a snap lock or external standard latches to lock the first and second side walls 22, 24 against each other in a closed position. In another embodiment, the vessel 14 may have a side wall of different configuration, for example, a square shaped side wall instead of a cylindrical side wall. It should be noted herein that the vessel 14 discussed herein in an exemplary embodiment and should not be construed as a limitation of the scope of the disclosure. Other suitable designs of the vessel are also envisioned within the scope of the disclosure.

The vessel 14 may include one or more flexible heater pads (not shown) provided on an inner surface of the cylindrical side wall 20. The flexible heater pads are configured to heat an enclosure when the enclosure is loaded within the vessel 14. In some embodiments, the flexible heater pads are provided symmetrically around the loaded enclosure. In one embodiment, the flexible heater pads are made of but not limited to silicone, polyimide, or other flexible heat-resistant polymers disposed enclosing electrical heating elements which typically are conductive fibers or films. In some embodiments, the vessel 14 can additionally include a flexible cooling jacket provided to the cylindrical side wall 20.

In one embodiment, the vessel 14 includes a sensor support (not shown) coupled to cylindrical side wall 20. In one particular embodiment, a rod may be provided along a the cylindrical side wall 20 of the vessel 14 and the sensor support is attached to the rod such that it can be slid along the rod in order to adjust a height position of the sensor support. The sensor support includes an elongated, horizontal rail onto which sensors can be mounted. Sensors, such as but not limited to, for example, PH and dissolved oxygen sensors may be mounted to the sensor support. In some embodiments, the sensor support may protrude outwards from the vessel 14. In certain embodiments, the sensor support may be rotatable about an attachment point to the rod.

As mentioned earlier, the base module 12 includes the base support 16 and the impeller drive unit 18 disposed within the base support 16. In one embodiment, the impeller drive unit 18 includes a motor.

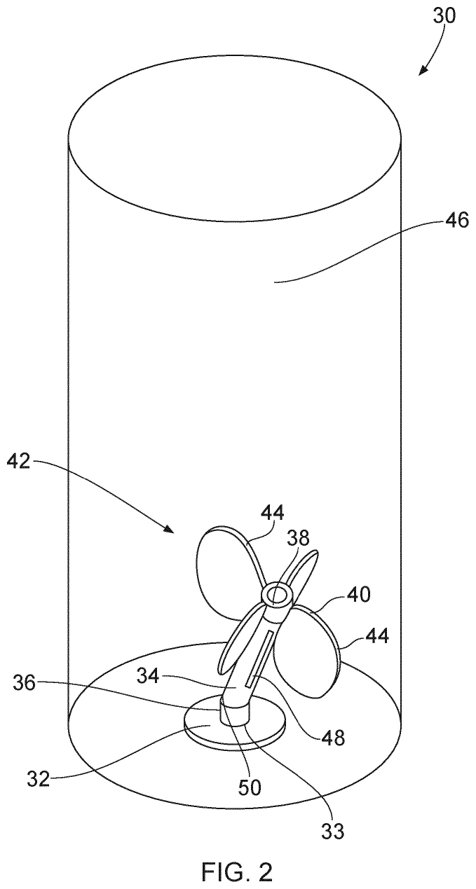

FIG. 2 is a schematic perspective view of a device 30 according to one embodiment of the present disclosure. The device 30 includes a base connector 32 having an opening 33. In one embodiment, the base connector 32 is a plate having the opening 33. The base connector 32 may be made of any suitable material depending on the application. In other embodiments, other types of base connector 32 may be envisioned. An impeller connector (not shown in FIG. 2) is coupled to the base connector 32. Specifically, the impeller connector includes a through-passage (not shown in FIG. 2) aligned with the opening 33 of the base connector 32.

Further, the device 30 includes a flexible tube 34 having a first end 36 coupled to the impeller connector. The flexible tube 34 may be made of any suitable material which provides flexibility properties. Further, the device 30 includes a seal component 38 coupled to a second end 40 of the flexible tube 34. As result, the flexible tube 34 is a sealed flexible tube. In another embodiment, the second end 40 of the flexible tube may be sealed by welding. Additionally, an impeller 42 is coupled to the second end 40 of the flexible tube 34. The impeller 42 includes a plurality of rotatable vanes 44 which can either be rotatable along a clockwise direction or an anticlockwise direction. In one embodiment, the rotatable blades 44 are flat rotatable blades located along a vertical direction. In another embodiment, the rotatable blades 44 are located at an inclined angle, for example, 45 degrees with reference to a vertical axis. In yet another embodiment, each of the rotatable blades 44 include a leading face which can either be flat or concave shaped, whereas back sides are convex shaped. The design of the rotatable blades 44 may vary depending on the application.

Further, the device 30 includes an enclosure 46 disposed enclosing the impeller 42, the flexible tube 34, the impeller connector, and the base connector 32. In one embodiment, the enclosure 46 is a disposable bag used in a bioreactor. In one embodiment, the enclosure 46 is a pre-sterilized bag. In another embodiment, the enclosure 46 may be a container. In one embodiment, the vessel 14 may also include a sparger located below with reference to the impeller 42, for aeration of a medium filled inside the enclosure 46. The agitation of the medium provided by the impeller 42 facilitates distribution of air bubbles emanating from the sparger.

In one embodiment, the device 30 includes a friction reduction component 48 disposed on an inner peripheral surface 50 of the flexible tube 34. In one example, the friction reduction component 48 is a lubrication coating applied on the inner peripheral surface 50 of the flexible tube 34. In other examples, other types of friction reduction components such as bearings may also envisioned. The assembly of the device 30 along with the vessel 14 and the drive shaft are explained in detail with reference to subsequent figures.

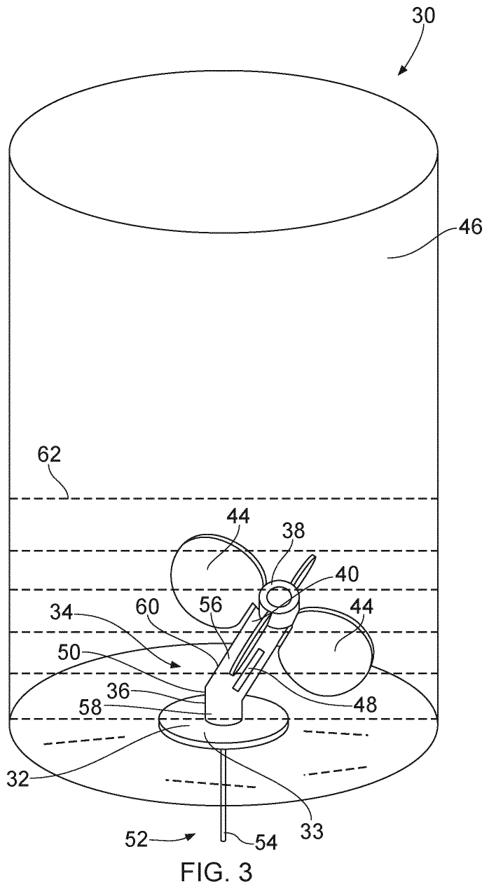

FIG. 3 is a schematic perspective view of the device 30 according to one embodiment of the present disclosure. As discussed previously, the device 30 includes the impeller connector (not shown in FIG. 3) coupled to the base connector 32. Further, the first end 36 of the flexible tube 34 is coupled to the impeller connector. Further, the seal component 38 is coupled to the second end 40 of the flexible tube 34. Additionally, the impeller 42 having the plurality of vanes 44 is coupled to the second end 40 of the flexible tube 34. Further, the bag 46 disposed enclosing the impeller 42, the flexible tube 34, the impeller connector, and the base connector 32.

In the illustrated embodiment, a drive shaft 52 of the base module 12 (shown in FIG. 1) is also shown. The drive shaft 52 includes a straight portion 54 and a bend portion 56. It should be noted herein that the bend portion 56 is inclined at a predefined angle with reference to the straight portion 54. The drive shaft 52 extends through the base connector 32, the impeller connector, and the flexible tube 34. Specifically, the drive shaft 52 extends through the opening 33 of the base connector 32, the through-passage of the impeller connector, and the flexible tube 34.

The flexible tube 34 conforms to the profile of the drive shaft 52 because the flexible tube 34 has elastic properties. Specifically, the flexible tube 34 has a first portion 58 coupled to the impeller connector and a second portion 60 disposed enclosing the bend portion 56 of the drive shaft 52. The first portion 58 of the flexible tube 34 conforms to the profile of the impeller connector and the straight portion 54 of the drive shaft 52 whereas the second portion 60 of the flexible tube 34 conforms to the profile of the bend portion of the drive shaft 52. Herein, more specifically, the impeller 42 is coupled to the bend portion 56 of the drive shaft 52 via the second end 40 of the flexible tube 34.

Further, the enclosure 46 is filled with a medium 62 such as but not limited to a culture medium used in a bioreactor. In such an embodiment, the enclosure 46 may be a pre-sterilized bag, for example, a bag pre-sterilized by gamma radiation. It should be noted herein that the flexible tube 34 is a sealed tube and hence prevents contact of the medium 62 filled in the enclosure 46 with the drive shaft 52.

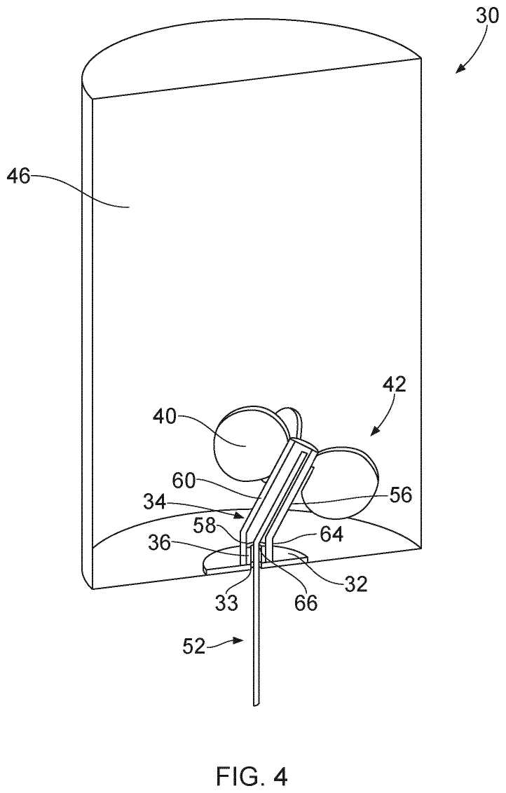

FIG. 4 is a partial schematic perspective view of the device 30 according to one embodiment of the present disclosure. In the illustrated embodiment, the device 30 includes the impeller connector 64 coupled to the base connector 32. In one embodiment, the impeller connector 64 is integrated to the base connector 32 to form a single molded component during manufacture. In another embodiment, the impeller connector 64 and the base connector 32 are separate components and detachably coupled to each other. Further, the first end 36 of the flexible tube 34 is coupled to the impeller connector 64. Additionally, the impeller 42 is coupled to the second end 40 of the flexible tube 34. Further, the enclosure 46 is disposed enclosing the impeller 42, the flexible tube 34, the impeller connector 64, and the base connector 32.

In the illustrated embodiment, the drive shaft 52 extends through the base connector 32, the impeller connector 64, and the flexible tube 34. Specifically, the drive shaft 52 extends through the opening 33 of the base connector 32, the through-passage 66 of the impeller connector 64, and the flexible tube 34.

Specifically, the flexible tube 34 has the first portion 58 coupled to the impeller connector 64 and the second portion 60 disposed enclosing the bend portion 56 of the drive shaft 52. In one embodiment, the bend portion 56 of the drive shaft 52 contacts the inner peripheral surface 50 of the flexible tube 34. It should be noted herein that the first portion 58 of the flexible tube 34 is not rotatable because the first portion 58 is coupled to the impeller connector 64, whereas the second portion 60 of the flexible tube 34 is rotatable along with the drive shaft 52 and the impeller 42. Herein, more specifically, the impeller 42 is coupled to the bend portion 56 of the drive shaft 52 via the second end 40 of the flexible tube 34.

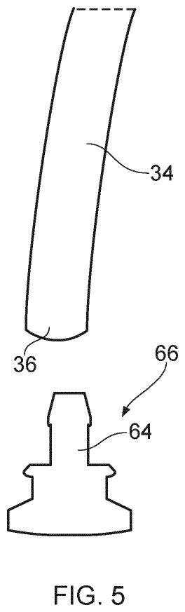

FIG. 5 is a schematic perspective view of the flexible tube 34 and the impeller connector 64 according to the embodiment of FIG. 4 of the present disclosure. In the illustrated embodiment, the first end 36 of the flexible tube 34 is coupled to the impeller connector 64. In one embodiment, the impeller connector 64 is but not limited to a tube barb. In other embodiments, other types of impeller connectors are also envisioned.

As discussed earlier, the drive shaft 52 extends through the base connector 32, the impeller connector 64, and the flexible tube 34. Specifically, the drive shaft 52 extends through the opening 33 of the base connector 32, the through-passage 66 of the impeller connector 64, and the flexible tube 34.

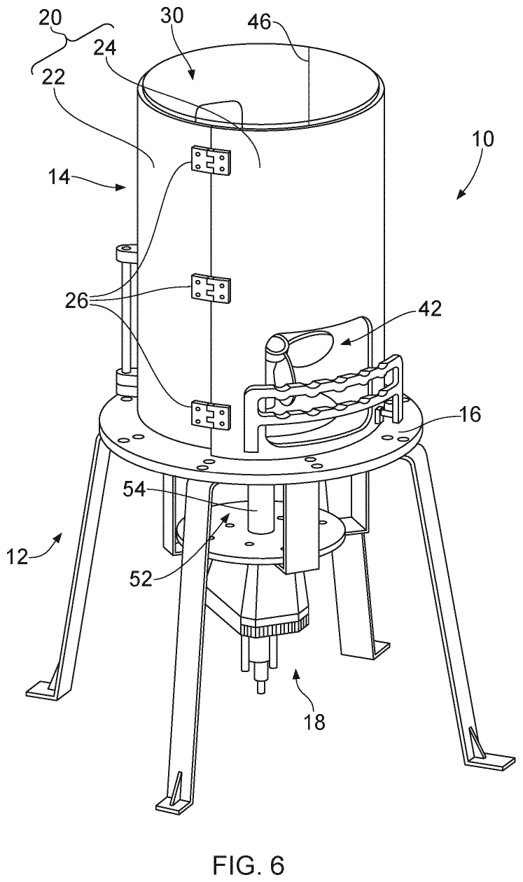

FIG. 6 is a schematic perspective view of the system 10 according to the embodiments FIGS. 1-5 of the present disclosure. As discussed earlier, the system 10 include the base module 12 and the vessel 14 which are configured to support and substantially enclose the enclosure 46 of the device 30. The base module 12 includes the base support 16 and the impeller drive unit 18 disposed within the base support 16. The vessel 14 and the device 30 are stably supported by the base support 16. The vessel 14 includes the cylindrical side wall 20 having the first side wall 22 and the second side wall 24 coupled to each other via the plurality of hinges 26. The second side wall 24 can be opened to access interior of the vessel 14 and for loading and unloading the device 30 having the enclosure 46.

As mentioned earlier, the device 30 includes the enclosure 46 disposed enclosing the impeller 42, the flexible tube 34, the impeller connector 64, and the base connector 32. In one embodiment, the enclosure 46 is a disposable bag used in a bioreactor. In another embodiment, the enclosure 46 may be a container. The drive shaft 52 extends through the base connector 32, the impeller connector 64, and the flexible tube 34. Specifically, the drive shaft 52 extends through the opening 33 of the base connector 32, the through-passage 66 of the impeller connector 64, and the flexible tube 34. The straight portion 54 of the drive shaft 52 has a coupler which is directly coupled to the impeller drive unit 18. In one embodiment, the straight portion 54 of the drive shaft 52 is substantially perpendicular to the base support 16.

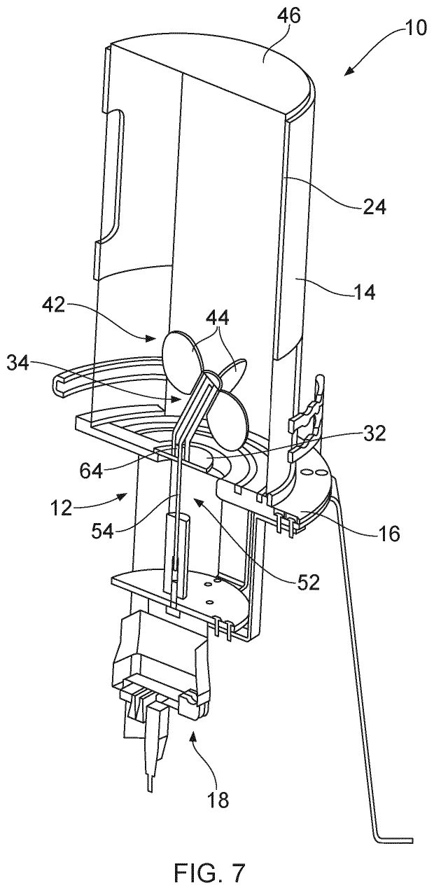

FIG. 7 is a partial schematic perspective view of the system 10 according to the embodiments of FIGS. 1-6 of the present disclosure. As discussed earlier, the system 10 includes the base module 12 and the vessel 14 which are configured to support and substantially enclose the enclosure 46 of the device 30. The base module 12 includes the base support 16 and the impeller drive unit 18 disposed within the base support 16. The vessel 14 and the device 30 are stably supported by the base support 16. The second side wall 24 can be opened to access interior of the vessel 14 and for loading and unloading the device 30 having the enclosure 46. As mentioned earlier, the device 30 includes the enclosure 46 disposed enclosing the impeller 42, the flexible tube 34, the impeller connector 64, and the base connector 32. The drive shaft 52 extends through the base connector 32, the impeller connector 64, and the flexible tube 34. The straight portion 54 of the drive shaft 52 is directly coupled to the impeller drive unit 18.

During operation of the system 10, the impeller drive unit 18 is powered by a power source. In one embodiment, the impeller drive unit 18 includes a motor. As a result, the impeller drive unit 18 drives the impeller 42 via the drive shaft 52. The impeller 42 having the plurality of rotatable blades 44 is used to stir the medium 62 such as, for example, culture medium filled inside the enclosure 46. It should be noted herein that the first portion 58 of the flexible tube 34 is not rotatable because the first portion 58 is coupled to the impeller connector 64, whereas the second portion 60 of the flexible tube 34 is rotatable along with the drive shaft 52 and the impeller 42. In accordance with the embodiments of the present disclosure, the straight portion 54 of drive shaft 52 rotates around a substantially vertical axis, causing the bent portion 56 of the drive shaft 52, the second end 40 of the flexible tube 34, and the impeller 42 to gyrate in a horizontal plane around the substantially vertical axis. Also, the flexible tube 34 prevents contact of the medium 62 with the drive shaft 52 because the drive shaft 52 is enclosed by the sealed flexible tube 34. Also, even if the bend portion 56 of the drive shaft 52 contacts the inner peripheral surface 50 of the flexible tube 34, the friction reduction component 48 enables to reduce the friction between the bend portion 56 of the drive shaft 52 and the flexible tube 34. Furthermore, since the bend portion 56 of the drive shaft 52 is inclined at a predefined angle relative the straight portion 54 of the drive shaft 52, the contact area of the impeller 42 with the medium 62 is enhanced during stirring process. As a result, the agitation of the medium 62 within the enclosure 46 is also enhanced.

In accordance with the embodiments discussed herein, the exemplary system 10 has an impeller drive unit which is directly coupled to a drive shaft for transmitting drive motion to an impeller. As a result, the system 10 does not need additional bearings and magnets for transmitting the drive motion compared to a convention magnetic stirrer-based agitator. Hence, the exemplary system 10 has fewer and less frictional parts compared to a conventional system. Also, the flexible tube prevents contact of the medium with the drive shaft because the drive shaft is enclosed by the sealed flexible tube. Hence, contamination due to impeller parts is minimized. It should be noted herein that although bioreactors are mostly discussed herein, the exemplary system 10 may be applicable to any application where there is a requirement to prevent contact of a stirring medium to the drive shaft.

1. A device comprising:
a base connector having an opening;
an impeller connector coupled to the base connector, wherein the impeller connector has a through-passage aligned with the opening of the base connector;
a flexible tube having a first end and a second end, wherein the first end of the flexible tube is coupled to the impeller connector;
a seal component coupled to the second end of the flexible tube;
an impeller coupled to the second end of the flexible tube; and
an enclosure disposed enclosing the impeller, the flexible tube, the impeller connector, and the base connector.

2. The device as claimed in claim 1, wherein the impeller connector is a tube barb.

3. The device as claimed in claim 1, further comprising a friction reduction component disposed on an inner peripheral surface of the flexible tube.

4. The device as claimed in claim 1, wherein the impeller comprises one or more rotatable vanes.

5. The device as claimed in claim 1, wherein the enclosure is a pre-sterilized bag.

6. The device as claimed in claim 1, wherein the base connector and the impeller connector are integrated to form a single molded component.

7. The device as claimed in claim 1, wherein the base connector and the impeller connector are detachably coupled to each other.

8. A system comprising:
a base module comprising:
a base support;
an impeller drive unit disposed within the base support; and
a drive shaft having a straight portion and a bend portion, wherein the straight portion is directly coupled to the impeller drive unit;
a device comprising:
a base connector having an opening, disposed on and coupled to the base support;
an impeller connector coupled to the base connector, wherein the impeller connector has a through-passage aligned with the opening of the base connector;
a flexible tube having a first end and a second end, wherein the first end of the flexible tube is coupled to the impeller connector; and wherein the drive shaft extends through the opening of the base connector, the through-passage of the impeller connector, and the flexible tube;
seal component coupled to the second end of the flexible tube;
an impeller coupled to the bend portion of the drive shaft via the second end of the flexible tube; and
an enclosure coupled to the base support and disposed enclosing the bend portion of the drive shaft, the impeller, the flexible tube, the impeller connector, and the base connector.

9. The system as claimed in claim 8, wherein the impeller drive unit comprises a motor.

10. The system as claimed in claim 8, wherein the straight portion of the drive shaft is disposed substantially perpendicular to the base support.

11. The system as claimed in claim 8, wherein the impeller connector is a tube barb.

12. The system as claimed in claim 8, wherein the bend portion of the drive shaft, comprises an end which contacts an inner peripheral surface of the flexible tube.

13. The system as claimed in claim 8, wherein the device further comprises a friction reduction component disposed on an inner peripheral surface of the flexible tube.

14. The system as claimed in claim 8, wherein the enclosure is a pre-sterilized bag.

15. The system as claimed in claim 14, further comprising a vessel coupled to the base support and disposed enclosing the pre-sterilized bag.

16. The system as claimed in claim 15, wherein the system is a bioreactor, and wherein the bag is filled with a culture medium.

17. A method comprising:
driving an impeller of a device by an impeller drive unit of a base module via a drive shaft;
wherein the drive shaft comprises a straight portion and a bend portion, wherein the straight portion is directly coupled to the impeller drive unit, wherein the device further comprises a base connector coupled to an impeller connector which is further coupled to a first end of a sealed flexible tube, and wherein the drive shaft extends through an opening of the base connector, a through-passage of the impeller connector, and the flexible tube, and wherein the impeller is coupled to the bend portion of the drive shaft via a second end of the sealed flexible tube;
stirring a medium filled inside an enclosure, by the impeller;
wherein a portion of the sealed flexible tube enclosing the bend portion of the drive shaft rotates along with the impeller and the drive shaft.

18. The method as claimed in claim 17, further comprising preventing a contact of the medium with the drive shaft, by the sealed flexible tube.

19. The method as claimed in claim 17, further comprising reducing a friction between the bend portion of the drive shaft and an inner peripheral surface of the flexible tube by a friction reduction component.

20. The method as claimed in claim 17, wherein the medium is a culture medium of a bioreactor.
